(19) United States
(12) Patent Application Publication
(10) Pub. No.: US 2008/0020475 A1
(43) Pub. Date: Jan. 24, 2008

(54) METHODS AND KITS FOR THE DIAGNOSIS OF HYPOTHYROIDISM

(75) Inventors: Jim Mapes, Austin, TX (US); Michael Spain, Austin, TX (US); Ralph McDade, Austin, TX (US); Kenneth Pass, Glenmont, NY (US)

Correspondence Address: FOLEY AND LARDNER LLP;SUITE 500, 3000 K STREET NW, WASHINGTON, DC, 20007, US

(73) Assignees: Rules-Based Medicine, Inc; Health Research, Inc.

(21) Appl. No.: 11/744,723

(22) Filed: May 4, 2007

Related U.S. Application Data
(60) Provisional application No. 60/797,673, filed on May 5, 2006.

(51) Int. Cl. G01N 33/53; G06F 15/18

(52) U.S. Cl. 436/87; 706/13

(57) ABSTRACT

Provided are methods for the detection and diagnosis of Hypothyroidism. The methods are based on the discovery that altered levels of selected analytes in sample fluid, typically blood samples, of patients are supportive of a diagnosis of Hypothyroidism. At least twenty-four new biomarkers for hypothyroidism are thus disclosed (singly or in any combination), Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, or Herpes Simplex Virus 1 Glycoprotein D Antibody. Altogether the concentrations of one or more of these analytes, as well as Thyroid Stimulating Hormone, or any combination thereof, provide a sensitive and selective picture of the patient's condition, namely, whether the patient is suffering from Hypothyroidism. Kits containing reagents to assist in the analysis of fluid samples are also described.

### CROSS-REFERENCE TO RELATED APPLICATIONS

This application claims priority to U.S. Provisional Application No. 60/797,673 filed May 5, 2006, the disclosure of which is incorporated herein by reference in its entirety.

### FIELD OF THE INVENTION

Methods, kits, and reagents for detection and/or diagnosis of Hypothyroidism.

### DESCRIPTION OF THE RELATED ART

Hypothyroidism is a common medical condition, affecting up to seventeen percent of women over age sixty. Although it is more common in middle-aged and older women, anyone can develop the condition, including males, infants, and adolescents. The pathology of Hypothyroidism involves underproduction of hormones by the thyroid gland. Causes of Hypothyroidism include autoimmune disease (Hashimoto's thyroiditis), radioactive iodine treatment, radiation therapy, thyroid surgery, lithium and other medications, pituitary disorders or failure, pregnancy, iodine deficiency, and congenital defects (approximately one in three thousand babies is born with a defective or missing thyroid gland). Not every cause of Hypothyroidism is known. Symptoms of Hypothyroidism progress over time and include increased sensitivity to cold; constipation; pale, dry skin; puffy face; hoarse voice; elevated blood cholesterol; weight gain; muscle aches, tenderness, stiffness, and weakness; joint pain, stiffness, and swelling; heavier menstruation; and depression.

If untreated, Hypothyroidism can lead to more severe health problems including goiter, heart problems, mental health problems (depression, decreased libido), myxedema, and birth defects. The standard treatment for Hypothyroidism is daily oral administration of synthetic thyroid hormone. Treatment is typically successful if the patient's Thyroid Stimulating Hormone (“TSH”) level is monitored to maintain the correct replacement therapy dosage (increased production of TSH is the body's attempt at stimulating an under active thyroid gland, and therefore TSH level provides a useful indicator of proper thyroid function). As Hypothyroidism is a chronic condition, treatment is usually lifelong. Diagnosis of Hypothyroidism is based upon risk factors (sex, age), clinical symptoms, and a blood test for the level of TSH and sometimes the thyroid hormones T-3 and/or T-4 (the “TSH/T-4 test”). These facts provide the primary rationale for improving the screening and diagnosis of patients for Hypothyroidism.

The selectivity of current assays for congenital Hypothyroidism in newborns are lacking, as the current TSH/T-4 based test yields an 80-89% false positive rate Thus, there is a critical need to develop biomarkers for detection of congenital Hypothyroidism and the attendant need to administer treatments.

### SUMMARY OF THE INVENTION

A method for rapid detection and/or accurate diagnosis of Hypothyroidism is provided. The method can be practiced with a determination of the concentrations of one or more biomarkers in a patient fluid sample. Elevated (or depressed, as the case might be) levels of the one or more biomarkers, which are statistically different from levels found in “normals” (that is, control subjects not suffering from Hypothyroidism), support a positive diagnosis of Hypothyroidism. Preferably, the method utilizes a panel of analytes or “biomarkers,” up to ten or more substances found in a sample fluid (e.g., whole blood, serum, plasma, or urine, or the fluid sample is obtained from a spot of whole blood, serum, plasma, or urine), to help support a positive or negative diagnosis of Hypothyroidism. In certain embodiments, up to 99% accuracy in making a correct diagnosis is provided by the method, including when used to supplement the current standard TSH/T-4 testing.

According to the invention, a method of diagnosing Hypothyroidism in a human subject who is clinically normal, exhibiting clinical symptoms of Hypothyroidism, or analytically positive for hypothyroidism based on TSH/T-4 testing is provided, which comprises: (a) obtaining a fluid sample from such a human subject; (b) determining the concentrations of at least one of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, or Prostate-Specific Antigen, or any combination thereof; (c) comparing the determined concentrations of the at least one of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, or Prostate-Specific Antigen, or any combination thereof, in said fluid sample against reference ranges for each biomarker, which comparison provides an indication for a positive or negative diagnosis of Hypothyroidism. The human subject can be an adult, juvenile, tot, newborn, or pre-natal subject and can be male or female.

In another embodiment of the invention, a method of diagnosing Hypothyroidism in a human male subject who is clinically normal, exhibiting clinical symptoms of Hypothyroidism, or analytically positive for hypothyroidism based on TSH/T-4 testing is provided, which comprises: (a) obtaining a fluid sample from such a human subject; (b) determining the concentrations of at least one of Insulin, Thyroid Stimulating Hormone, Interleukin-4, Granulocyte Macrophage Colony Stimulating Factor, Matrix Metalloproteinase 9, Interleukin-12p40, Calcitonin, Tissue Factor, or Lymphotactin, or any combination thereof; (c) comparing the determined concentrations of the at least one of Insulin, Thyroid Stimulating Hormone, Interleukin-4, Granulocyte Macrophage Colony Stimulating Factor, Matrix Metalloproteinase 9, Interleukin-12p40, Calcitonin, Tissue Factor, or Lymphotactin in said fluid sample against reference ranges for each biomarker, which comparison provides an indication for a positive or negative diagnosis of Hypothyroidism.

In still another embodiment of the invention, a method of diagnosing Hypothyroidism in a human male subject who is clinically normal, exhibiting clinical symptoms of Hypothyroidism, or analytically positive for hypothyroidism based on TSH/T-4 testing is provided, which comprises: (a) obtaining a fluid sample from such a human subject; (b) determining the concentrations of at least one of Thyroid Stimulating Hormone, Matrix Metalloproteinase 9, Double Stranded DNA Antibody, Proliferating Cell Nuclear Antigen Antibody, Calcitonin, Tissue Factor, Smith Antibody, or Herpes Simplex Virus 1 Glycoprotein D Antibody, or any combination thereof; (c) comparing the determined concentrations of the at least one of Thyroid Stimulating Hormone, Matrix Metalloproteinase 9, Double Stranded DNA Antibody, Proliferating Cell Nuclear Antigen Antibody, Calcitonin, Tissue Factor, Smith Antibody, or Herpes Simplex Virus 1 Glycoprotein D Antibody in said fluid sample against reference ranges for each biomarker, which comparison provides an indication for a positive or negative diagnosis of Hypothyroidism.

In still another embodiment of the invention, a method of diagnosing Hypothyroidism in a human female subject who is clinically normal, exhibiting clinical symptoms of Hypothyroidism, or analytically positive for hypothyroidism based on TSH/T-4 testing is provided, which comprises: (a) obtaining a fluid sample from such a human subject; (b) determining the concentrations of at least one of Immunoglobulin E, Thyroid Stimulating Hormone, Tumor Necrosis Factor Alpha, Fatty Acid Binding Protein, Insulin, Prostate-Specific Antigen, Alpha Fetoprotein, Growth Stimulating Hormone, Alpha-2 Macroglobulin, or Interleukin-12p40, or any combination thereof; (c) comparing the determined concentrations of the at least one of Immunoglobulin E, Thyroid Stimulating Hormone, Tumor Necrosis Factor Alpha, Fatty Acid Binding Protein, Insulin, Prostate-Specific Antigen, Alpha Fetoprotein, Growth Stimulating Hormone, Alpha-2 Macroglobulin, or Interleukin-12p40 in said fluid sample against reference ranges for each biomarker, which comparison provides an indication for a positive or negative diagnosis of Hypothyroidism.

In still another embodiment of the invention, a method of diagnosing Hypothyroidism in a human female subject who is clinically normal, exhibiting clinical symptoms of Hypothyroidism, or analytically positive for hypothyroidism based on TSH/T-4 testing is provided, which comprises: (a) obtaining a fluid sample from such a human subject; (b) determining the concentrations of at least one of Thyroid Stimulating Hormone, Serum Glutamic Oxaloacetic Transaminase, Tissue Factor, Tumor Necrosis Factor Alpha, Matrix Metalloproteinase 3, Cancer Antigen 125, Fatty Acid Binding Protein, Alpha-2 Macroglobulin, Prostate-Specific Antigen, or Mumps Antibody, or any combination thereof; (c) comparing the determined concentrations of the at least one of Thyroid Stimulating Hormone, Serum Glutamic Oxaloacetic Transaminase, Tissue Factor, Tumor Necrosis Factor Alpha, Matrix Metalloproteinase 3, Cancer Antigen 125, Fatty Acid Binding Protein, Alpha-2 Macroglobulin, Prostate-Specific Antigen, or Mumps Antibody in said fluid sample against reference ranges for each biomarker, which comparison provides an indication for a positive or negative diagnosis of Hypothyroidism.

Various techniques for assessing the importance of certain biomarkers in arriving at a diagnosis are also described herein. One such technique is a projection of compiled results on a proximity map, whereby the proximity of a subject's determined concentrations to a cluster of other subjects' determined concentrations, who were previously diagnosed as having suffered from Hypothyroidism, contributes to a positive diagnosis of Hypothyroidism. Other techniques include the application of one or more statistical methods (e.g., linear regression analysis, classification tree analysis, heuristic nave Bayes analysis and the like).

Also provided is a kit comprising reagents for determining the concentration in a fluid sample of a panel of analytes including one or more of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, or Herpes Simplex Virus 1 Glycoprotein D Antibody, or any combination thereof. The reagents may include antibodies against the members of a given panel of analytes. Furthermore, the reagent may be immobilized on a substrate, which substrate may comprise a two-dimensional array, a microtiter plate, or multiple bead sets.

The methods may further comprise comparing the levels of one or more of the biomarkers mentioned above in a patient's blood with levels of the same biomarkers in one or more groups of one or more control samples by applying a statistical method such as: linear regression analysis, classification tree analysis, and heuristic nave Bayes analysis. Groups of control samples may include (i) normal subjects, (ii) subjects analytically positive for Hypothyroidism based on the TSH/T-4 and/or other tests but clinically negative for Hypothyroidism (“False Positives”), or (iii) subjects analytically positive for Hypothyroidism based on the TSH/T-4 and/or other tests and clinically positive for Hypothyroidism (“True Positives”). The statistical method may be, and typically is performed by a computer process, such as by commercially available statistical analysis software. In one embodiment, the statistical method is a classification tree analysis, for example CART (Classification and Regression Tree). Results for a particular patient or subject, whose sample fluid is tested against a panel of biomarkers according to the method, can be projected onto a proximity map. The proximity of a particular patient's biomarker concentration results to one of at least two control populations (those previously diagnosed as suffering from Hypothyroidism and normals) supports a either a positive or negative diagnosis of Hypothyroidism. Such algorithms classify complex spectra from biological materials, such as a blood sample, to distinguish individuals as normal or as possessing biomarker expression levels characteristic of a particular disease state. While such algorithms may be used to increase the speed and efficiency of the application of the decision rule and to avoid investigator bias, one of ordinary skill in the art will realize that computer-based algorithms are not required to carry out the methods of the present invention.

An article of manufacture is provided which comprises binding reagents specific for at least one of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, or Herpes Simplex Virus 1 Glycoprotein D Antibody, or any combination thereof. In a preferred embodiment, each binding reagent is immobilized on a substrate. For example, monoclonal antibodies against Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, or Herpes Simplex Virus 1 Glycoprotein D Antibody, or any combination thereof, are immobilized independently to one or more discrete locations on one or more surfaces of one or more substrates. The substrates may be beads comprising an identifiable biomarker, wherein each binding reagent is attached to a bead comprising a different identifiable biomarker than beads to which a different binding reagent is attached. The identifiable biomarker may comprise a fluorescent compound, a quantum dot, or the like.

Other aspects of the invention will become apparent to those of ordinary skill after considering the detailed descriptions provided herewith.

### BRIEF DESCRIPTION OF THE DRAWINGS

FIG. 1 is a projection of a proximity map of three groups of patients whose fluid samples were tested against a panel of biomarkers TSH, IL-12p40, TNF-α, Tissue Factor, IL-15, Insulin, IgE, GST, Calcitonin, and PSA among Normal (green or light gray), previously False Positive (yellow or white), and True Positive (red or dark gray) subjects. The results of this proximity map analysis indicate that consideration of the biomarkers listed provides the ability to distinguish true positive subjects from normal subjects. Furthermore, the number of subjects that were falsely positive under currently available diagnostic methods is reduced in the method provided herein.

FIG. 2 is a projection of a proximity map of three groups of male only patients whose fluid samples were tested against a panel of biomarkers Insulin, TSH, IL-4, GM-CSF, MMP-9, IL-12-p40, Calcitonin, Tissue Factor, and Lymphotactin among Normal (green or light gray), previously False Positive (yellow or white), and True Positive (red or dark gray) male subjects. The results of this proximity map analysis indicate that consideration of the biomarkers listed provides the ability to distinguish true positive subjects from normal subjects. Furthermore, the number of subjects that were falsely positive under currently available diagnostic methods is reduced in the method provided herein.

FIG. 3 is a projection of a proximity map of three groups of female only patients whose fluid samples were tested against a panel of biomarkers IgE, TSH, NF-α, FABP, Insulin, PSA, α-Fetoprotein, GST, α-2 macroglobulin, and IL-12p40 among Normal (green or light gray), previously False Positive (yellow or white), and True Positive (red or dark gray) female subjects. The results of this proximity map analysis indicate that consideration of the biomarkers listed provides the ability to distinguish true positive subjects from normal subjects. The number of subjects that were falsely positive under currently available diagnostic methods is reduced in the present method, although there was an increase in the number of subjects that were falsely negative in the present method over currently available diagnostic methods.

FIG. 4 is a projection of a proximity map of two groups of female only patients whose fluid samples were tested against a panel of biomarkers TSH, SGOT, Tissue Factor, TNF-α, MMP3, CA125, FABP, α-2 Macroglobulin, PSA, and Mumps Ab between previously False Positive (yellow or white) and True Positive (red or dark gray) female subjects. The results of this proximity map analysis indicate that consideration of the biomarkers listed provides the ability to distinguish true positive subjects from subjects that were falsely positive under currently available diagnostic methods.

FIG. 5 is a projection of a proximity map of two groups of male only patients whose fluid samples were tested against a panel of biomarkers TSH, MMP-9, dsDNA Ab, PCNA Ab, Calcitonin, Tissue Factor, Smith Ab, and HSV-1 gd Ab between previously False Positive (yellow or white) and True Positive (red or dark gray) male subjects. The results of this proximity map analysis indicate that consideration of the biomarkers listed may provide the ability to distinguish true positive subjects from subjects that were falsely positive under currently available diagnostic methods.

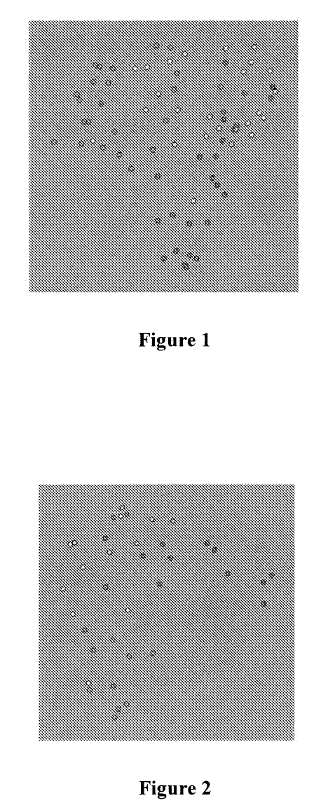

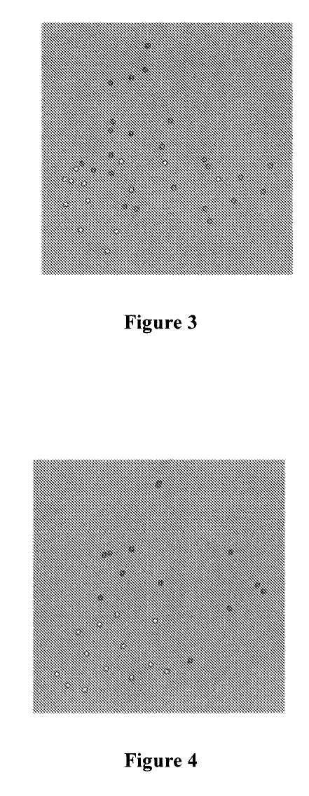

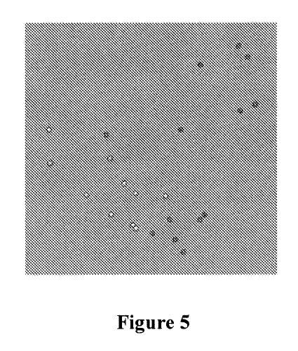

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Provided herein are multifactorial assays for diagnosis of Hypothyroidism in a patient or patients. Identified below are certain sample fluid (e.g., blood) analytes or biomarkers useful in the detection and/or diagnosis of Hypothyroidism. It has been found that each of the following biomarkers either is over-expressed or is under-expressed in the blood or other sample fluids of patients with clinically confirmed Hypothyroidism.

Identified as being useful in the detection or proper diagnosis of subjects suffering from Hypothyroidism are the biomarkers Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and Herpes Simplex Virus 1 Glycoprotein D Antibody, or any combination thereof.

The parameters for establishing the significance of one or more biomarkers for the diagnosis of Hypothyroidism are determined statistically by comparing normal or control blood (preferably, e.g., serum or plasma) levels of these biomarkers with blood levels in patients clinically and properly diagnosed as suffering from Hypothyroidism. Statistical methods can be used to define the critical range of values. Typically within one standard deviation of those approximate values might be considered as statistically significant values for determining a statistically significant difference, preferably two standard deviations. For this reason, the word “about” is used in connection with any stated values herein.

“Statistical classification methods” are used to identify biomarkers capable of discriminating normal patients from patients with Hypothyroidism and are further used to determine critical blood values for each biomarker for discriminating between such patients. Certain statistical methods can be used to identify discriminating biomarkers and panels thereof. These statistical methods may include, but are not limited to: 1) linear regression; 2) classification tree methods; and 3) statistical machine learning to optimize the unbiased performance of algorithms for making predictions. Each of these statistical methods is well-known to those of ordinary skill in the field of biostatistics and can be performed as a process in a computer. A large number of software products are available commercially to implement statistical methods, such as, without limitation, S-PLUS™, commercially available from Insightful Corporation of Seattle, Wash.

In one embodiment, comparison of an individual's biomarker profile to a reference biomarker profile comprises applying a decision rule. The decision rule can comprise a data analysis algorithm, such as a computer pattern recognition algorithm. Other suitable algorithms include, but are not limited to, logistic regression or a nonparametric algorithm that detects differences in the distribution of feature values (e.g., a Wilcoxon Signed Rank Test). The decision rule may be based upon one, two, three, four, five, 10, 20 or more features. In one embodiment, the decision rule is based on hundreds or more of features. Applying the decision rule may also comprise using a classification tree algorithm. For example, the reference biomarker profile may comprise at least three features, where the features are predictors in a classification tree algorithm. The data analysis algorithm predicts membership within a population (or class) with an accuracy of at least about 60%, preferably at least about 70%, more preferably at least about 80% and most preferably at least about 90%. Suitable algorithms are known in the art, any of which may be suitable for application with the instant invention.

By identifying biomarkers useful in the determination and/or diagnosis of Hypothyroidism and by use of statistical methods to identify which biomarkers and groups of biomarkers are particularly useful in identifying Hypothyroidism patients, a person of ordinary skill in the art, based on the disclosure herein, can compose panels of biomarkers having superior selectivity and sensitivity. Examples of biomarkers that can be included in panels, which provide discriminatory capability, include: Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and Herpes Simplex Virus 1 Glycoprotein D Antibody. Examples of specific panels comprising selected biomarkers from the above-mentioned list, include, but are not limited to: TSH, IL-12p40, TNF-α, Tissue Factor, IL-15, Insulin, IgE, GST, Calcitonin, and PSA; (ii) Insulin, TSH, IL-4, GM-CSF, MMP-9, IL-12-p40, Calcitonin, Tissue Factor, and Lymphotactin; (iii) IgE, TSH, TNF-α, FABP, Insulin, PSA, α-Fetoprotein, GST, α-2 macroglobulin, and IL-12p40; (iv) TSH, SGOT, Tissue Factor, TNF-α, MMP3, CA125, FABP, α-2 Macroglobulin, PSA, and Mumps Ab; and (v) SH, MMP-9, dsDNA Ab, PCNA Ab, Calcitonin, Tissue Factor, Smith Ab, and HSV-1 gd Ab. It will be recognized by those of ordinary skill in the field of biostatistics, that the number of biomarkers in any given panel may vary depending on the combination of biomarkers. With optimum sensitivity and specificity being the goal, one panel may include two biomarkers, another may include five, and still others may include eight, nine, ten, or more, yielding similar results.

The invention is based on an evaluation of levels of at least Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, or Herpes Simplex Virus 1 Glycoprotein D Antibody, or any combination thereof, in serum for diagnosis of Hypothyroidism in human subjects at all stages of development beginning in utero. Patients with Hyopthyroidism suffer numerous symptoms, and outcomes can be improved with appropriate diagnosis and therapy. Thus, accurate diagnosis of patients suspected of suffering from Hypothyroidism is critical for patient care.

The present method includes, but is not limited to, measuring the level of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and/or Herpes Simplex Virus 1 Glycoprotein D Antibody in a biological sample (e.g., whole blood, plasma, serum or urine and the like) from a patient; comparing the respective levels with that of control subjects; and diagnosing the state of disease based on the level of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and/or Herpes Simplex Virus 1 Glycoprotein D Antibody relative to that of control subjects. A patient can be diagnosed with Hypothyroidism if the level of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and/or Herpes Simplex Virus 1 Glycoprotein D Antibody is increased or decreased relative to that of control subjects.

Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and Herpes Simplex Virus 1 Glycoprotein D Antibody can be captured with anti-Thyroid Stimulating Hormone, anti-Interleukin-12p40, anti-Tumor Necrosis Factor Alpha, anti-Tissue Factor, anti-Interleukin-15, anti-Insulin, anti-Immunoglobulin E, anti-Growth Stimulating Hormone, anti-Calcitonin, anti-Prostate-Specific Antigen, anti-Interleukin-4, anti-Granulocyte Macrophage Colony Stimulating Factor, anti-Matrix Metalloproteinase 9, anti-Lymphotactin, anti-Fatty Acid Binding Protein, anti-Alpha Fetoprotein, anti-Alpha-2 Macroglobulin, anti-Serum Glutamic Oxaloacetic Transaminase, anti-Matrix Metalloproteinase 3, anti-Cancer Antigen 125, anti-Mumps Antibody, anti-Double Stranded DNA Antibody, anti-Proliferating Cell Nuclear Antigen Antibody, anti-Smith Antibody, and anti-Herpes Simplex Virus 1 Glycoprotein D Antibody, respectively, or with corresponding monoclonal antibodies. The diagnostic method may include measuring the levels of one or more of these analytes relative to that of control subjects.

Analyte levels can be measured using an immunoassay such as an ELISA or a multiplexed method as described below, and in more detail by Chandler et al., U.S. Pat. No. 5,981,180 (Luminex Corporation). All patents referenced herein are incorporated in their entirety by reference.

Altered Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and Herpes Simplex Virus 1 Glycoprotein D Antibody levels were identified in Hypothyroidism patients. Thus, these analytes are valuable biomarkers, potentially identifying Hypothyroidism patients, and potentially without the false positive result that are common with current methods of diagnosis. The role Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, or Herpes Simplex Virus 1 Glycoprotein D Antibody may play in the pathophysiology of Hypothyroidism is unknown.

The analytes used in the method of the invention can be detected, for example, by a binding assay. For example, a sandwich immunoassay can be performed by capturing Interleukin-12p40 and Tumor Necrosis Factor Alpha from a biological sample with antibodies having specific binding affinity for each protein, which then can be detected with a labeled antibody having specific binding affinity for each analyte. Alternatively, standard immunohistochemical techniques can be used to detect Interleukin-12p40 and Tumor Necrosis Factor Alpha using such antibodies. Antibodies having affinity for Interleukin-12p40 and Tumor Necrosis Factor Alpha, as well as for the rest of the analytes listed above, are generally available.

The term “binding reagent” and like terms, refers to any compound, composition or molecule capable of specifically or substantially specifically (that is with limited cross-reactivity) binding another compound or molecule, which, in the case of immune-recognition is an epitope. The binding reagents typically are antibodies, preferably monoclonal antibodies, or derivatives or analogs thereof, but also include, without limitation: Fᵥ fragments; single chain Fᵥ (scFᵥ) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fᵥ fragments, scFᵥ tandems ((scFᵥ)₂ fragments), diabodies, tribodies or tetrabodies, which typically are covalently linked or otherwise stabilized (i.e., leucine zipper or helix stabilized) scFᵥ fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very low affinity and cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475096, 5,840867 and 6,544,776.

The ELISA and Luminex LabMAP immunoassays described below are examples of sandwich assays. The term “sandwich assay” refers to an immunoassay where the antigen is sandwiched between two binding reagents, which are typically antibodies—the first binding reagent/antibody being attached to a surface and the second binding reagent/antibody comprising a detectable group. Examples of detectable groups include, for example and without limitation: fluorochromes, enzymes, or epitopes for binding an additional binding reagent (for example, when the second binding reagent/antibody is a mouse antibody, such an epitope is detectable by an additional fluorescently labeled anti-mouse antibody), such as an antigen or member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822, and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present invention, but they are not preferred. As used herein, “immunoassays” refer to immune assays, typically, but not exclusively sandwich assays, capable of detecting and quantifying a desired blood biomarker, namely at least one of Thyroid Stimulating Hormone, Interleukin-12p40, Tumor Necrosis Factor Alpha, Tissue Factor, Interleukin-15, Insulin, Immunoglobulin E, Growth Stimulating Hormone, Calcitonin, Prostate-Specific Antigen, Interleukin-4, Granulocyte Macrophage Colony Stimulating Factor, Matrix Metalloproteinase 9, Lymphotactin, Fatty Acid Binding Protein, Alpha Fetoprotein, Alpha-2 Macroglobulin, Serum Glutamic Oxaloacetic Transaminase, Matrix Metalloproteinase 3, Cancer Antigen 125, Mumps Antibody, Double Stranded DNA Antibody, Proliferating Cell Nuclear Antigen Antibody, Smith Antibody, and Herpes Simplex Virus 1 Glycoprotein D Antibody, or any combination thereof.

In the context of the present disclosure, “blood” includes any blood fraction, for example serum, that can be analyzed according to the methods described herein. Serum is a standard blood fraction that can be tested, and is tested in the Examples below. By measuring blood levels of a particular biomarker, it is meant that any appropriate blood fraction can be tested to determine blood levels and that data can be reported as a value present in that fraction. As a non-limiting example, the blood levels of a biomarker can be presented as 50 pg/mL serum.

### EXAMPLE 1

Patient Population. The patient population was chosen based on having Hypothyroidism confirmed by a clinician trained and experienced in diagnosing same, based upon the presence of physical attributes and symptoms of Hypothyroidism. The sample of blood, which was tested, was obtained (e.g., upon visit to a doctor). The normal or control patient population was chosen from a wellness clinic. These control patients had no indication of suffering from Hypothyroidism. Another group of patients was chosen based upon having a positive TSH/T-4 Hypothyroid test but having a negative clinical diagnosis for Hypothyroidism. Consent and blood specimens from all participants were obtained under IRB Protocol.

Spotting and processing of blood specimens. Dried blood spot specimens were clinical specimens collected by applying a few drops of blood, freshly drawn by finger stick with a lancet from adults, or by heel stick with a lancet from infants, onto specially manufactured absorbent specimen collection (filter) paper. The blood was allowed to saturate the paper and was air dried for a minimum of 3 hours. Caked or clotted specimens were not desirable and therefore not shipped. The specimen collection technique and the specifications for specimen matrix and shipment have been published as a national standard by the National Committee for Clinical Laboratory Standards.

Specimen collection materials (“collection kits”) for newborn screening may include a sturdy paper overlay that covers the absorbent filter paper containing the dried specimen. These are then enclosed and sealed in a high quality bond envelope. The paper overlay and the sealed bond envelope provide a double-layer barrier that protects casual handlers (i.e., shipping handlers and other nonlaboratory, non-technical personnel) from accidental exposure to the dried blood specimens and protects the specimens from exposure to the environment during shipping.

The dried blood spot specimens should not be packaged in airtight, leak-proof plastic bags, because the lack of air exchange in the inner environment of a sealed plastic bag has been found to cause heat buildup and moisture accumulation that can damage the dried blood spot test substances. In addition, various chemicals that can adversely affect the test substances in the dried blood spots can leach from these plastics and thus cause incorrect analytical test results. The inclusion of desiccant packs can aid in prevention of moisture accumulation, but shipping conditions are typically uncontrolled and desiccant has a limited effectiveness.

Once received, the dried blood spot specimens are subject to various analytical procedures. The specimen is screened for congenital and inherited metabolic disorders among the more than 4.2 million infants born annually in the United States. Efficient collection, shipment, and analysis of dried blood spot specimens on filter paper comprises the foundation of this important public health service. Other applications include DNA (genetic) analyses, forensic studies, immunologic studies, and nutritional evaluations of infants, children, and adults.

Example to the Procedure in one embodiment of the invention follows:
1. Obtain the filter paper with blood spots from storage.
2. Remove enough separation devices and 1.5 ml centrifuge tubes to process the number of blood spots removed.
3. Determine if the blood stain covers an area where three holes (approx. 6.2 mm diameter) can be punched using the a single hole puncher. If not enough blood is covering the spot, notify supervisor for steps to follow.
4. Punch three holes per sample making sure that each punched hole is completely stained with blood.
5. Place the three spots into separation device and place the device into the 1.5 ml centrifuge tube. Lay the holes flat against the bottom of the separation device and label the tube with sample ID. Repeat procedure for each filter paper until all samples are ready for elution.
6. Add 130 μl elution buffer (4% BSA-PBS) to each separation device. Care should be exercised not to force liquid pass the separation media, thus shortening the volume for elution. Ensure that elution buffer is completely covering the blood spots. The caps should not be capped at this time.
7. Allow the spots to soak for at least 5 minutes at room temp.
8. Set vortex at low setting and carefully mix the elution buffer with spots. Avoid forceful shaking as liquid will pass thru separation device.
9. Cover tubes carefully using lab film. Do not cap the tubes as pressure will force liquid pass the separation device.
10. Place the covered centrifuge vials with separation devices at 2-8° C. for minimum 12 hours (overnight) elution.
11. Remove tubes from 2-8° C. and carefully remove cover film. Cap the tubes and place in centrifuge. Centrifuge the devices at 14 k RPM for 1 minute.
12. Remove the devices from centrifuge and inspect each tube for elution completion. Liquid should be collected at bottom of centrifuge tube and the eluted spots in the filter paper of the separation device. The spots should appear almost dried and with little to no traces of eluent.
13. Remove the separation device from 1.5 ml centrifuge tube and discard.

Recap the tube.
14. Return the eluted samples in the tubes to 2-8° C. until ready for further processing. The final dilution of the sample after elution is 1:7.

Development of Luminex assay. The reagents for multiplex system were developed using antibody pairs purchased from R&D Systems (Minneapolis, Minn.), Fitzgerald Industries International (Concord, Mass.) or produced by well known immunological methods. Capture antibodies were monoclonal and detection antibodies were polyclonal. Capture Abs were covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking.

Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4° C. Coupling efficiency of monoclonal antibodies was tested by staining 2,000 microspheres with PE-conjugated goat anti-mouse IgG. Detection Abs were biotinylated using EZ-Link Sulfo-NHS-Biotinylation Kit according to manufacturer's protocol. The extent of biotin incorporation was determined using HABA assay and was 20 moles of biotin per mole of protein. The assays were further optimized for concentration of detection Ab and for incubation times. Sensitivity of the newly developed assays were determined using serially diluted purified proteins. Intra-assay variability, expressed as a coefficient of variation, was calculated based on the average for patient samples and measured twice at two different time points. The intra-assay variability within the replicates is expressed as an average coefficient of variation. Inter-assay variability was evaluated by testing quadruplicates of each standard and sample with an average of 16.5%. Newly developed kits were multiplexed together and the absence of cross-reactivity was confirmed according to Luminex protocol.

Additionally, Cancer Antigen 125 reagent for multiplex system was developed using antibody pair purchased from Fitzgerald Industries International. Capture antibody was monoclonal and detection antibody was sheep polyclonal. Capture Ab was biotinylated using EZ-Link Sulfo-NHS-Biotinylation Kit according to the manufacturer's protocol. The extent of biotin incorporation was determined using HABA assay and was 20 moles of biotin per mole of protein. Capture Ab was covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath for 2 min. An aliquot of 2.5×10⁶ microspheres was resuspended in microtiter tubes containing 0.1 M sodium phosphate buffer, pH 6.1 (phosphate buffer), to a final volume of 80 μL.

This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride, both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C. Coupling efficiency of monoclonal antibodies was tested by staining 2,000 microspheres with PE-conjugated goat anti-mouse IgG. The assay was further optimized for concentration of detection Ab and for incubation times.

Sensitivity of the newly developed assay as determined in a Luminex assay using serially diluted purified CA-125, was 20 IU. Intra-assay variability, expressed as a coefficient of variation, was calculated based on the average for patient samples and measured twice at at least two different time points. The intra-assay variability within the replicates presented as an average coefficient of variation was 8.5%. Interassay variability was evaluated by testing quadruplicates of each standard and 10 samples. The variabilities of these samples were between 10 and 22%, with an average of 16.5%. Next, the anti-CA-125 microspheres were combined with the existing multiplex kit.

Statistical Analysis of Data. All statistical analyses were conducted using S-Plus statistical software (Seattle, Wash.: Math Soft, Inc., 1999). The data were first randomly split into a training and test set. Logistic regression (Hosmer, D W, S Lemeshow, Applied Logistic Regression. New York, N.Y.: John Wiley & Sons, 1989) was then used to calculate the optimal weighting of each biomarker and the subsequent predicted probability of being a case. All predicted probabilities ≧0.5 were categorized as a predicted case; predicted probabilities <0.5 were categorized as a predicted control. After fitting a logistic model to the training set, classification of disease status was then calculated for the test set.

Results

Serum concentrations of biomarkers by LabMap technology. Circulating concentrations of different serum biomarkers were evaluated in a multiplexed assay using LabMap technology in blood of patients from Hypothyroidism and both control groups.

Proximity Map Analysis. The proximity map data analysis is conducted with a software program that groups samples by their similarities in analyte concentration patterns. A unique chemical signature is generated using the concentration of the analytes measured in each sample. The relationship of each sample signature is visualized in the Galaxy™ projection. The Galaxy™ is a proximity map, such that the closer two objects are in the visualization, the closer their chemical signatures are, and thus the more similar they are to one another. The axes are dimensionless (a result of being derived from a principal component analysis), and thus the visualization is not a typical X-Y scatter plot in which moving along one axis means increasing or decreasing a single value. The two axes of the Galaxy™ are defined by the first two principal components, a common method to reduce complex data. The placement of objects (record points) is done using a set of heuristics that have been designed to maximize the preservation of spatial relationships that existed in the high-dimensional space of the original data while minimizing the overlap that can occur when doing simple projections.

An examination of the FIGs, shows that the red circles (dark gray, the Hypothyroidism patients) are separated from the green circles (light gray, the negative controls) to various degrees with all of the plots attaining separation. FIG. 4 provides what is possibly the best separation. If an unknown sample is tested for the analytes listed in FIG. 4, then the location of the patient (from whom the unknown sample is taken) on the plot would be indicative of whether that patient has Hypothyroidism or not, greatly reducing the prospect of a false positive result over the current TSH/T-4 test alone. The area where the two clusters adjoin would be an indeterminate area.

Rates of classification accuracy (in discriminating controls from Fragile X patients) were then obtained using 10-fold cross-validation, and a Receiver Operating Characteristic (ROC) curve was generated. The sensitivity and specificity of the method depend on the cut-point (i.e. predicted probability from the classification tree) used to classify each subject as either a case or control.

### EXAMPLE 2

Development of LabMAP Assays for Circulating Antibodies. Assays were performed in filter-bottom 96-well microplates. Purified antigens of interest were coupled to Luminex beads as described for antibodies. Antigen-coupled beads were pre-incubated with blocking buffer containing 4% BSA for 1 h at room temperature on microtiter shaker. Beads were then washed three times with washing buffer (PBS, 1% BSA, 0.05% Tween 20) using a vacuum manifold followed by incubation with 50 μL blood serum diluted 1:250 for 30 min at 4° C. This dilution was selected as an optimal for recovery of anti-IL-18 IgG based on previous serum titration. Next, washing procedure was repeated as above and beads were incubated with 50 μL/well of 4 μg/mL PE-conjugated antibody raised against human IgG, for 45 min in the dark with constant shaking. Wells were washed twice, assay buffer was added to each well and samples were analyzed using the Bio-Plex suspension array system. For standard curve, antigen-coupled beads were incubated with serially diluted human antibodies against specific antigens. Purification of monospecific human antibodies is described above. Data analysis was performed using five-parametric-curve fitting.

Whereas particular embodiments of the invention have been described herein for the purpose of illustrating the invention and not for the purpose of limiting the same, it will be appreciated by those of ordinary skill in the art that numerous variations of the details, materials, and arrangement of parts may be made within the principle and scope of the invention without departing from the invention as described in the appended claims.

What is claimed is:

1. A method of generating a test panel for diagnosing hypothyroidism in a human subject suspected of suffering from hypothyroidism, comprising:
(a) obtaining fluid samples from at least two groups of human subjects: at least one of which is populated by human subjects suspected of suffering from hypothyroidism and at least one of which is populated by human subjects representing a control group;
(b) determining for each fluid sample a concentration of each of a plurality of biomarkers;
(c) identifying from the results of step (b) those biomarkers whose concentrations differ statistically between the at least two groups of human subjects; and
(d) incorporating at least some of the biomarker concentrations identified in step (c) in a test panel, against which concentrations of biomarkers found in a fluid sample from a test subject can be compared in an effort to arrive at a diagnosis for hypothyroidism.

2. The method of claim 1 in which the human subject is a neonate.

3. The method of claim 1 in which said fluid sample is selected from the group consisting of whole blood, plasma, serum, or urine, or in which said fluid sample is obtained from a spot of whole blood, plasma, serum, or urine.

4. The method of claim 1 in which the at least some of the biomarker concentrations identified in step (c) are selected from the concentrations of TSH, Insulin, IL-12p40, IgE, TNF-α, GST, Tissue Factor, Calcitonin, IL-15, PSA, and various combinations thereof.

5. The method of claim 1 in which the at least some of the biomarker concentrations identified in step (c) are selected from the concentrations of TSH, Insulin, IL-12p40, Calcitonin, IL-4, Tissue Factor, GM-CSF, Lymphotactin, MMP-9, and various combinations thereof.

6. The method of claim 1 in which the at least some of the biomarker concentrations identified in step (c) are selected from the concentrations of TSH, Insulin, IL-12p40, IgE, PSA, α-Fetoprotein, TNF-α, GST, FABP, α-2 macroglobulin, and various combinations thereof.

7. The method of claim 1 in which the at least some of the biomarker concentrations identified in step (c) are selected from the concentrations of TSH, CA125, SGOT, FABP, Tissue Factor, α-2 Macroglobulin, TNFα, PSA, MMP3, Mumps Ab, and various combinations thereof.

8. The method of claim 1 in which the at least some of the biomarker concentrations identified in step (c) are selected from the concentrations of TSH, Calcitonin, MMP9, Tissue Factor, dsDNA Ab, Smith Ab, PCNA Ab, HSV-1 gd Ab, and various combinations thereof.

9. The method of claim 1 in which the concentration of each of the plurality of biomarkers is determined by a binding assay.

10. The method of claim 9 in which the binding assay is carried out using a flow analyzer.

11. The method of claim 1 in which the at least two groups of human subjects are of the same sex.

12. The method of claim 1 in which the control group is negative for hypothyroidism.

13. The method of claim 1 in which the control group is analytically positive but clinically negative for hypothyroidism.

14. The method of claim 1 in which at least one group is analytically positive but clinically negative for hypothyroidism.

15. The method of claim 1 in which at least one group is analytically and clinically positive for hypothyroidism.

16. A method for attempting a positive or negative diagnosis of hypothyroidism in a human subject suspected of suffering from hypothyroidism, comprising:
(a) obtaining a fluid sample from a human subject suspected of suffering from hypothyroidism;
(b) determining a concentration in the fluid sample for each member of a test panel, the test panel comprising biomarkers whose concentrations have been found to be statistically different between at least two groups of human subjects: at least one of which is populated by human subjects suffering from hypothyroidism and at least one of which is populated by human subjects who do not suffer from hypothyroidism;
(c) comparing at least some of the biomarker concentrations determined in step (b) against reference ranges associated with specific biomarkers, which comparison provides an indication for a positive or negative diagnosis of hypothyroidism in the human subject.

17. The method of claim 16 in which the test panel comprises biomarkers selected from TSH, Insulin, IL-12p40, IgE, TNF-α, GST, Tissue Factor, Calcitonin, IL-15, PSA, and various combinations thereof.

18. The method of claim 16 in which the test panel comprises biomarkers selected from TSH, Insulin, IL-12p40, Calcitonin, IL-4, Tissue Factor, GM-CSF, Lymphotactin, MMP-9, and various combinations thereof.

19. The method of claim 16 in which the test panel comprises biomarkers selected from TSH, Insulin, IL-12p40, IgE, PSA, TSH, α-Fetoprotein, TNF-α, GST, FABP, α-2 macroglobulin, and various combinations thereof.

20. The method of claim 16 in which the test panel comprises biomarkers selected from TSH, CA125, SGOT, FABP, Tissue Factor, α-2 Macroglobulin, TNFα, PSA, MMP3, Mumps Ab, and various combinations thereof.

21. The method of claim 16 in which the test panel comprises biomarkers selected from TSH, Calcitonin, MMP9, Tissue Factor, dsDNA Ab, Smith Ab, PCNA Ab, HSV-1 gd Ab, and various combinations thereof.

22. The method of claim 17 in which the at least one group is populated by human subjects analytically and clinically suffering from hypothyroidism and the at least one other group is populated by human subjects who are negative for hypothyroidism.

23. The method of claim 22 in which the at least one other group is comprised at least in part of human subjects who are analytically positive for hypothyroidism but clinically negative for hypothyroidism.

24. The method of claim 18 in which the at least one group is populated by male human subjects analytically and clinically suffering from hypothyroidism and the at least one other group is populated by male human subjects who are negative for hypothyroidism.

25. The method of claim 24 in which the at least one other group is comprised at least in part of male human subjects who are analytically positive for hypothyroidism but clinically negative for hypothyroidism.

26. The method of claim 19 in which the at least one group is populated by female human subjects analytically and clinically suffering from hypothyroidism and the at least one other group is populated by female human subjects who are negative for hypothyroidism.

27. The method of claim 26 in which the at least one other group is comprised at least in part of female human subjects who are analytically positive for hypothyroidism but clinically negative for hypothyroidism.

28. The method of claim 20 in which the at least one group is populated by female human subjects analytically and clinically suffering from hypothyroidism and the at least one other group is populated by female human subjects who are analytically positive for hypothyroidism but clinically negative for hypothyroidism.

29. The method of claim 21 in which the at least one group is populated by male human subjects analytically and clinically suffering from hypothyroidism and the at least one other group is populated by male human subjects who are analytically positive for hypothyroidism but clinically negative for hypothyroidism.

30. The method of claim 28 in which the female human subject is analytically positive for TSH, T4 or both.

31. The method of claim 29 in which the male human subject is analytically positive for TSH, T4 or both.

32. The method of claim 1 in which a heuristic algorithm is used to identify those biomarkers whose concentrations differ statistically between the at least two groups of human subjects.

33. The method of claim 32 in which the heuristic algorithm is utilized in a computer software program.

34. The method of claim 33 in which the computer software program is OmniViz™.

35. The method of claim 16 in which the reference ranges are established based on concentrations found in a control group populated by human subjects who do not suffer from hypothyroidism

36. A kit for the diagnosis of hypothyroidism in a human comprising reagents for determining the presence in a fluid sample of each member of a test panel, the test panel comprising biomarkers whose presence or absence have been found to be statistically different between at least two groups of human subjects: at least one of which is populated by human subjects suffering from hypothyroidism and at least one of which is populated by human subjects who do not suffer from hypothyroidism.

37. A method of diagnosing hypothyroidism in a human subject suspected of suffering from hypothyroidism, comprising:
(a) obtaining a fluid sample from a human subject suspected of suffering from hypothyroidism;
(b) determining the concentrations of three or more biomarkers selected from at least three of TSH, Insulin, IL-12p40, IgE, TNF-α, GST, Tissue Factor, Calcitonin, IL-15, PSA, IL-4, GM-CSF, Lymphotactin, MMP-9, α-Fetoprotein, TNF-α, GST, FABP, α-2 macroglobulin, CA125, SGOT, FABP, MMP-3, Mumps Ab, dsDNA Ab, Smith Ab, PCNA Ab, and HSV-1 gd Ab in said fluid sample;
(c) deciding if the determined concentrations of the three or more biomarkers in said fluid sample are statistically different from those found in a control group of human subjects,
whereby a finding of statistically different concentrations for at least two of the three or more determined biomarker concentrations supports a positive diagnosis of hypothyroidism.
